(19) United States
(12) Patent Application Publication
(10) Pub. No.: US 2026/0305963 A1
(43) Pub. Date: Oct. 8, 2026

(54) SPHYGMOMANOMETER FOR DETERMINING IRREGULAR PULSE WAVE AND OPERATION METHOD THEREOF

(71) Applicant: KOROT CO., LTD., Seoul (KR)

(72) Inventors: Ki Chul CHA, Seoul (KR); Sangjin LEE, Gyeonggi-do (KR); Jong Heun SHIN, Seoul (KR)

(73) Assignee: KOROT CO., LTD., Seoul (KR)

(21) Appl. No.: 19/476,741

(22) PCT Filed: Jun. 28, 2024
(86) PCT No.: PCT/KR2024/009069

§ 371 Date: Oct. 20, 2025

(30) Foreign Application Priority Data
Jul. 12, 2023 (KR) 10-2023-0090679

(51) Int. Cl. A61B 5/022; A61B 5/00

(52) U.S. Cl. CPC A61B 5/02225; A61B 5/02208; A61B 5/7264; A61B 5/7282; A61B 5/742; A61B 5/746; A61B 5/7475

(57) ABSTRACT

The present disclosure relates to a sphygmomanometer for determining an irregular pulse wave, and an operation method thereof. The sphygmomanometer according to an embodiment of the present disclosure may comprise a processor configured to: display, on a display, oscillometric signals sensed from an examinee while the pressure on the inside of a cuff surrounding a portion of the body of the examinee varies; determine whether an irregular pulse wave exists, on the basis of information on the intervals between oscillometric signals; and display, on the display, a visual mark for a region determined to be the irregular pulse wave in the oscillometric signals.

### TECHNICAL FIELD

The following description relates to a blood pressure measuring device for determining an irregular pulse wave and a method of operating the same.

### BACKGROUND ART

A method of measuring blood pressure is divided into invasive and noninvasive methods. The invasive method is the most accurate method, but the noninvasive method is mainly used due to reasons, such as pain or infection. The noninvasive method may be divided into auscultation and oscillometric methods, and both methods use a cuff. The auscultation method is a method of listening to sound energy with the ears using a stethoscope, wherein the sound energy is generated by turbulence that occurs during a blood flow movement according to the opening and closing of blood vessels. The oscillometric method is mainly used for a sphygmomanometer and has lower accuracy compared to the auscultation method.

An arrhythmia is an important measurement item to determine heart function and is widely used for health diagnosis by detecting a change in blood pressure. A regular pulse wave is generated as the myocardium pumps. However, when the pulse wave is not generated regularly, disappears, or is generated irregularly, it is called an arrhythmia.

### DISCLOSURE OF THE INVENTION

### Technical Goals

The present disclosure may provide a sphygmomanometer for determining an irregular pulse wave using an oscillometric signal and determining whether the irregular pulse wave is caused by an arrhythmia and a method thereof.

The present disclosure may provide a sphygmomanometer for simultaneously showing an oscillometric signal and Korotkoff sound and showing a visual mark in an area of the Korotkoff sound and an oscillometric signal in which an irregular pulse wave occurs.

However, the technical aspects are not limited to the aforementioned aspects, and other technical aspects may be present.

### Technical Solutions

A sphygmomanometer according to an embodiment of the present disclosure may include a processor configured to display, on a display, an oscillometric signal sensed from an examinee while internal pressure of a cuff that surrounds a body part of the examinee varies, determine whether an irregular pulse wave is present based on interval information of the oscillometric signal, and when the irregular pulse wave is present, display a visual mark on the display for an area determined to be the irregular pulse wave in the oscillometric signal.

The processor may be configured to display a Korotkoff sound included in a sound signal collected by a microphone while the internal pressure of the cuff varies, together with the oscillometric signal on the display, and display the visual mark on an area of the Korotkoff sound corresponding to the area determined to be the irregular pulse wave in the oscillometric signal.

The processor may be configured to obtain the interval information based on cycles corresponding to a peak interval or a valley interval of the oscillometric signal.

The processor may be configured to obtain the interval information based on cycles corresponding to a peak interval or a valley interval of a graph that differentiates the oscillometric signal.

The processor may be configured to, when a cycle that deviates from a mean value of the cycles by a threshold or more is present among the cycles, determine that the irregular pulse wave is present.

The processor may be configured to determine a reference line for a peak value or a valley value of the oscillometric signal, and when an interval in which the peak value or the valley value deviates from the reference line by a threshold or more is present, not determine whether the irregular pulse wave is present in the interval.

The processor may be configured to, when the irregular pulse wave is present, determine whether the irregular pulse wave is cause by an arrhythmia, and when the irregular pulse wave is caused by the arrhythmia, display an alert on the display.

The processor may be configured to determine whether the irregular pulse wave is caused by atrial fibrillation of the arrhythmias by inputting the interval information to a machine learning model for determining the atrial fibrillation.

The processor may be configured to determine whether the irregular pulse wave is caused by premature contraction of the arrhythmias by inputting the interval information to a machine learning model for determining the premature contraction.

A sphygmomanometer according to an embodiment of the present disclosure may include a processor configured to display, on a display, an oscillometric signal sensed from an examinee while internal pressure of a cuff that surrounds a body part of the examinee varies, determine whether an irregular pulse wave is present based on interval information of the oscillometric signal, and when the irregular pulse wave is present, display an alert on the display by determining whether the irregular pulse wave is caused by an arrhythmia.

A method of operating a sphygmomanometer according to an embodiment of the present disclosure may include displaying, on a display, an oscillometric signal sensed from an examinee while internal pressure of a cuff that surrounds a body part of the examinee varies, determining whether an irregular pulse wave is present based on interval information of the oscillometric signal, and when the irregular pulse wave is present, displaying a visual mark on the display for an area determined to be the irregular pulse wave in the oscillometric signal.

The method may further include displaying a Korotkoff sound together with the oscillometric signal on the display, wherein the Korotkoff sound is included in a sound signal collected by a microphone while the internal pressure of the cuff varies, wherein the display of the visual mark on the display may include displaying the visual mark on an area of the Korotkoff sound corresponding to the area determined to be the irregular pulse wave in the oscillometric signal.

The method may further include obtaining the interval information based on cycles corresponding to a peak interval or a valley interval of the oscillometric signal.

The method may further include obtaining the interval information based on cycles corresponding to a peak interval or a valley interval of a graph that differentiates the oscillometric signal.

The determining of whether the irregular pulse wave is present may include, when a cycle that deviates from a mean value of the cycles by a threshold or more is present among the cycles, determining that the irregular pulse wave is present.

The determining of whether the irregular pulse wave is present may include determining a reference line for a peak value or a valley value of the oscillometric signal, and when an interval in which the peak value or the valley value deviates from the reference line by a threshold or more is present, not determining whether the irregular pulse wave is present in the interval.

The method may further include, when the irregular pulse wave is present, determining whether the irregular pulse wave is cause by an arrhythmia, and when the irregular pulse wave is caused by the arrhythmia, displaying an alert on the display.

The determining of whether the irregular pulse wave is caused by the arrhythmia may include determining whether the irregular pulse wave is caused by atrial fibrillation of the arrhythmias by inputting the interval information to a machine learning model for determining the atrial fibrillation.

The determining of whether the irregular pulse wave is caused by the arrhythmia may include determining whether the irregular pulse wave is caused by premature contraction of the arrhythmias by inputting the interval information to a machine learning model for determining the premature contraction.

A sphygmomanometer according to an embodiment of the present disclosure may include a processor configured to synchronize and display, on a display, two or more of an oscillometric signal sensed from an examinee while internal pressure of a cuff that surrounds a body part of the examinee varies, a Korotkoff sound included in a sound signal collected by a microphone while the internal pressure of the cuff varies, and an electrocardiogram measured from the examinee while the internal pressure of the cuff varies.

### Effects of the Invention

According to an embodiment of the present disclosure, a sphygmomanometer and method of providing health information to an examinee by determining an irregular pulse wave and an arrhythmia using interval information of oscillometric signals may be provided.

According to an embodiment of the present disclosure, a sphygmomanometer and method of allowing an examinee to intuitively recognize that an irregular pulse wave occurs by showing a visual mark in an area of Korotkoff sound and an oscillometric signal in which the irregular pulse wave occurs.

### BRIEF DESCRIPTION OF DRAWINGS

FIG. 1 is a diagram of a sphygmomanometer according to an embodiment of the present disclosure.

FIG. 2 is a flowchart of an operating method of a sphygmomanometer according to an embodiment of the present disclosure.

FIGS. 3A and 3B are diagrams of signals sensed from an examinee according to an embodiment of the present disclosure.

FIGS. 4 and 5 are diagrams of interval information of an oscillometric signal according to an embodiment of the present disclosure.

FIGS. 6A and 6B are diagrams of a measurement error according to an embodiment of the present disclosure.

FIGS. 7A and 7B are diagrams of displaying an irregular pulse wave detected from a signal sensed from an examinee according to an embodiment of the present disclosure.

FIG. 8 is a flowchart of an operating method of a sphygmomanometer according to an embodiment of the present disclosure.

FIG. 9 is a diagram of a sphygmomanometer according to an embodiment of the present disclosure.

### BEST MODE FOR CARRYING OUT THE INVENTION

Hereinafter, embodiments will be described in detail with reference to the accompanying drawings. The scope of the right, however, should not be construed as limited to the embodiments set forth herein. In the drawings, like reference numerals are used for like elements.

Various modifications may be made to the examples. Here, the examples are not construed as limited to the disclosure and should be understood to include all changes, equivalents, and replacements within the idea and the technical scope of the disclosure.

Although terms of “first” or “second” are used to explain various components, the components are not limited to the terms. These terms should be used only to distinguish one component from another component. For example, a first component may be referred to as a second component, and similarly, the second component may also be referred to as the first component.

The terminology used herein is for the purpose of describing particular examples only and is not to be limiting of the examples. As used herein, the singular forms “a”, “an”, and “the” are intended to include the plural forms as well, unless the context clearly indicates otherwise. As used herein, “A or B”, “at least one of A and B”, “at least one of A or B”, “A, B or C”, “at least one of A, B and C”, and “at least one of A, B, or C,” each of which may include any one of the items listed together in the corresponding one of the phrases, or all possible combinations thereof. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms including technical and scientific terms used herein have the same meaning as commonly understood by one of ordinary skill in the art to which the embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

When describing the examples with reference to the accompanying drawings, like reference numerals refer to like constituent elements and a repeated description related thereto will be omitted. In the description of embodiments, detailed description of well-known related structures or functions will be omitted when it is deemed that such description will cause ambiguous interpretation of the present disclosure.

Hereinafter, embodiments will be described in detail with reference to the accompanying drawings.

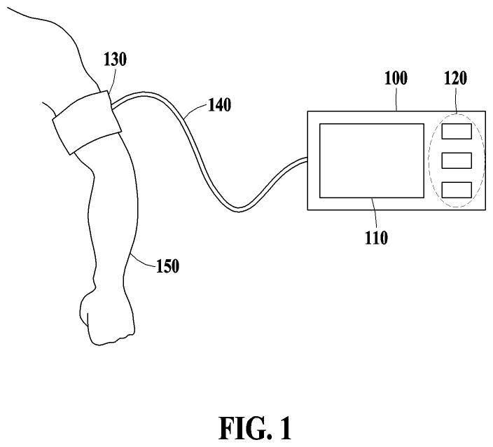

FIG. 1 is a diagram of a sphygmomanometer according to an embodiment of the present disclosure.

Referring to FIG. 1, a sphygmomanometer 100 may include a processor (not shown), a display 110, and an input unit 120.

The processor may control the overall operation of the sphygmomanometer 100 and may control other components included in the sphygmomanometer 100.

The display 110 may display a process and a result of blood pressure measurement. For example, the display 110 may display signals sensed from an examinee 150 in real time while internal pressure of a cuff 130 that surrounds a body part of the examinee 150 varies. When the blood pressure measurement is completed, the display 110 may display blood pressure information of the examinee 150. In addition, the display 110 may display various pieces of information related to blood pressure measurement without limitation.

The input unit 120 may receive an input related to blood pressure measurement from the examinee 150 and/or an examiner. For example, the input unit 120 may receive an input, such as start of operation of the sphygmomanometer 100, emergency stop, measured blood pressure information, or an output of pre-stored blood pressure information, but the example is not limited thereto. In FIG. 1, for ease of description, the input unit 120 is illustrated as a physical button. However, the example is not limited thereto, and the input unit 120 may be implemented in various forms (e.g., a touch, a jog dial, a switch, etc.). In addition, according to an embodiment, the input unit 120 may be omitted, and an input may be received from the examinee 150 and/or the examiner via the display 110 implemented as a touchscreen.

The cuff 130 may include an airbag that adjusts internal pressure by injecting fluid or discharging the injected fluid, and when measuring blood pressure, the airbag may surround a body part of the examinee 150. For example, the body part of the examinee 150 may be an upper arm, but is not limited thereto. Herein, the internal pressure of the airbag in the cuff 130 may also be referred to as internal pressure of the cuff 130 for ease of description.

A connecting line 140 may include a wire for connecting the sphygmomanometer 100 to the cuff 130 and a fluid tube. The wire may electrically connect a sensor (e.g., a microphone, a pressure sensor, etc.) in the cuff 130 to the sphygmomanometer 100. The fluid tube may transmit the fluid injected into the airbag in the cuff 130 or the fluid discharged from the airbag.

When the cuff 130 surrounds the upper arm of the examinee 150, the sphygmomanometer 100 may collect, using a microphone, a sound signal that occurs when the cuff 130 is pressurized to a level higher than typical systolic blood pressure and then is slowly depressurized and may sense an oscillometric signal from a pressure sensor. The sphygmomanometer 100 may detect Korotkoff sound from the sound signal by analyzing the sound signal and the oscillometric signal and may determine systolic blood pressure and diastolic blood pressure of the examinee 150 in real time based on the detected Korotkoff sound. In addition, while the sphygmomanometer 100 displays the detected Korotkoff sound on the display 110 in real time, when a reference Korotkoff sound corresponding to the systolic blood pressure and a reference Korotkoff sound corresponding to the diastolic blood pressure are displayed on the display 110 in real time, the sphygmomanometer 100 may display the reference Korotkoff sounds visually differently or may display visual marks (e.g., an arrow, a triangle, etc.) indicating the reference Korotkoff sounds.

As described above, the reliability of the measured blood pressure may be effectively improved for the examinee 150 by displaying information on the systolic blood pressure and diastolic blood pressure on the display 110 in real time when the Korotkoff sound detected while the cuff 130 is depressurized is displayed on the display 110 in real time. The examinee 150 may visually identify the detected Korotkoff sound in real time and may also visually identify the systolic blood pressure corresponding to a start point of the Korotkoff sound and the diastolic blood pressure corresponding to an end point of the Korotkoff sound, and accordingly, the reliability of a measurement result of the sphygmomanometer 100 for the examinee 150 may be improved.

According to an embodiment, the sphygmomanometer 100 may combine advantages of an auscultation method and an oscillometric method using characteristics of Korotkoff sound and an oscillometric signal (e.g., a correlation between the Korotkoff sound and the oscillometric signal, etc.). The sphygmomanometer 100 may sense an oscillometric signal. The sphygmomanometer 100 may determine whether a Korotkoff sound is detected in an interval based on a peak of an oscillometric signal and whether an amplitude of the Korotkoff sound is greater than or equal to a predetermined threshold amplitude. When a Korotkoff sound greater than or equal to the predetermined threshold amplitude is detected in the interval based on the peak of the oscillometric signal, the sphygmomanometer 100 may determine whether the Korotkoff sound is noise through a frequency analysis of the Korotkoff sound. When the sphygmomanometer 100 determines that the Korotkoff sound is not noise, the sphygmomanometer 100 may display the Korotkoff sound on the display 110 in real time.

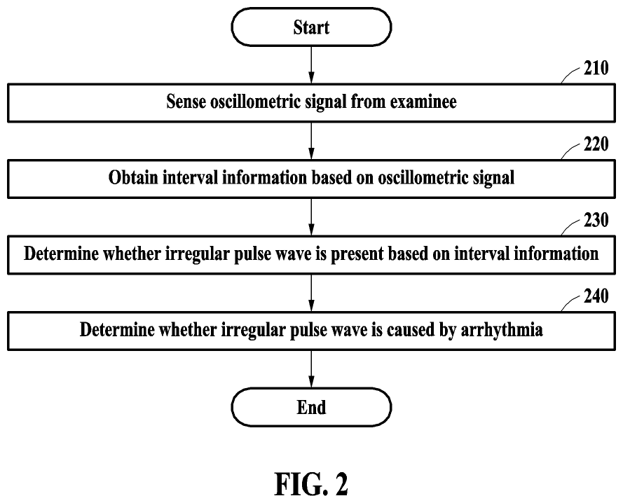

FIG. 2 is a flowchart of an operating method of a sphygmomanometer according to an embodiment of the present disclosure.

In the following embodiments, operations may be performed sequentially but not necessarily. For example, the order of the operations may change, and at least two of the operations may be performed in parallel. The operations shown in FIG. 2 may be performed by at least one component of the sphygmomanometer.

In operation 210, the sphygmomanometer may sense an oscillometric signal from an examinee.

The sphygmomanometer may sense the oscillometric signal from the examinee while internal pressure of a cuff that surrounds a body part of the examinee varies. In addition, the sphygmomanometer may detect a Korotkoff sound while sensing the oscillometric signal. The sphygmomanometer may display the oscillometric signal and the Korotkoff sound on a display in real time.

In operation 220, the sphygmomanometer may obtain interval information based on the oscillometric signal.

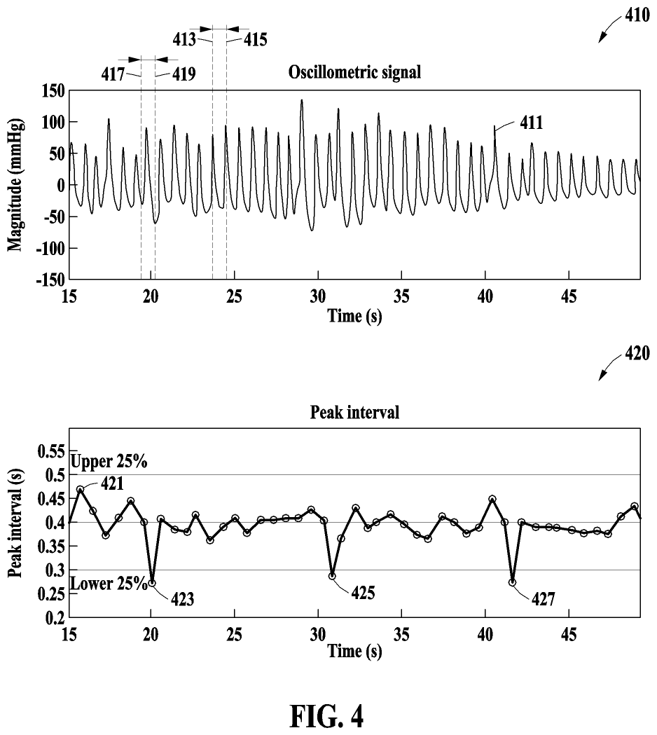

The sphygmomanometer may obtain the interval information based on the oscillometric signal after the blood pressure measurement is completed. The interval information may be information about an interval (in other words, a cycle) from a peak of an n-th signal of the oscillometric signal to a peak of an n+1-th signal. The interval information may be information about an interval (in other words, a cycle) from a valley of the n-th signal of the oscillometric signal to a valley of the n+1-th signal. An additional description of the interval information is provided with reference to FIG. 4.

In operation 230, the sphygmomanometer may determine whether an irregular pulse wave is present based on the interval information.

The sphygmomanometer may calculate a mean value of the interval information using pieces of interval information. When there is a cycle that deviates from the mean value of the interval information by a threshold value or more, the sphygmomanometer may determine that the irregular pulse wave is present.

In operation 240, the sphygmomanometer may determine whether the irregular pulse wave is caused by an arrhythmia.

When the irregular pulse wave is present, the sphygmomanometer may determine whether the irregular pulse wave is caused by the arrhythmia. Specifically, the sphygmomanometer may determine whether the irregular pulse wave is caused by an arrhythmia by inputting the information in which the irregular pulse wave is present to a machine learning model. The machine learning model may be a machine learning model trained to determine whether an irregular pulse wave is caused by an arrhythmia when the interval information is given. The machine learning model may correspond to a deep neural network (DNN), a convolutional neural network (CNN), and/or a recurrent neural network (RNN), and the like.

Hereinafter, the oscillometric signal and the Korotkoff sound displayed on the display of the sphygmomanometer are described.

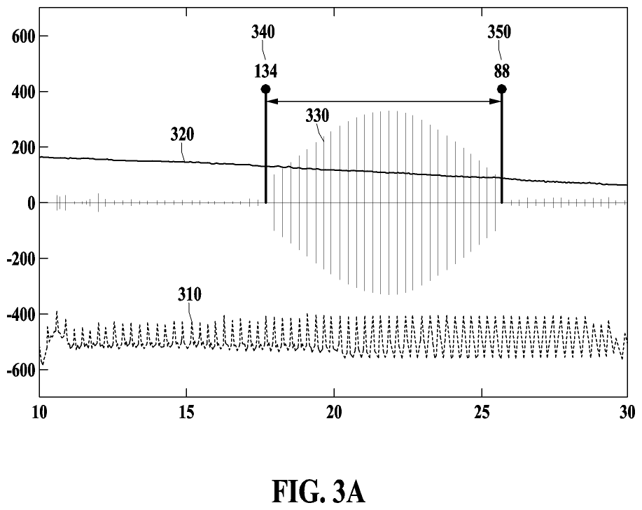

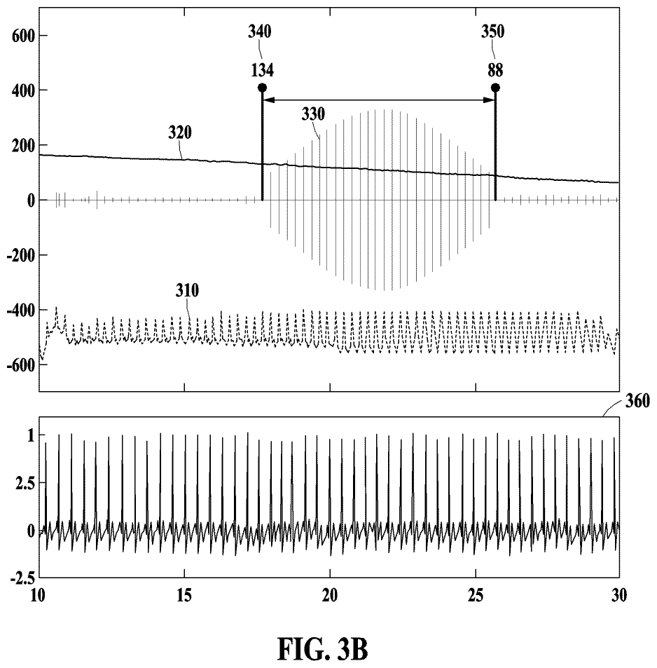

FIGS. 3A and 3B are diagrams of signals sensed from an examinee according to an embodiment of the present disclosure.

Referring to FIG. 3A, an oscillometric signal 310, a pressure signal 320, and a Korotkoff sound 330 are illustrated.

The oscillometric signal 310 may be sensed from the pressure signal 320. The oscillometric signal 310 may be extracted from the pressure signal 320 using a filter for extracting the oscillometric signal 310.

The pressure signal 320 may be a signal related to internal pressure of a cuff. An airbag in the cuff may be pressurized to measure blood pressure of an examinee and then may be slowly depressurized. In this case, arterial pressure caused by contraction from the heart may be transmitted to an upper arm. Accordingly, the arterial pressure may be transmitted to the airbag of the cuff that surrounds the upper arm. The pressure signal 320 may be a signal with respect to the internal pressure of the cuff and the arterial pressure. Accordingly, the pressure signal 320 may be shown as a straight line but may fluctuate depending on the arterial pressure.

The Korotkoff sound 330 may be a signal used for an auscultation method. The auscultation method may determine blood pressure based on the Korotkoff sound 330 that occurs when the cuff is pressurized and then is depressurized. For example, a point at which the Korotkoff sound 330 begins to occur when depressurizing the cuff may be determined to be systolic blood pressure (SBP), and a point at which the Korotkoff sound 330 no longer occurs and disappears may be determined to be diastolic blood pressure (DBP). The SBP may be referred to as highest blood pressure 340, and the DBP may be referred to as lowest blood pressure 350. Referring to FIG. 3A, the highest blood pressure 340 of the examinee may be 134 mmHg, and the lowest blood pressure 350 may be 88 mmHg. The Korotkoff sound 330 may be collected only between the highest blood pressure 340 and the lowest blood pressure 350.

On the other hand, the oscillometric signal 310 may be sensed outside the range between the highest blood pressure 340 and the lowest blood pressure 350, unlike the Korotkoff sound 330. Accordingly, of the oscillometric signal 310 herein, signals outside the range between the highest blood pressure 340 and the lowest blood pressure 350 may be used to obtain the interval information. This is because obtaining the interval information only between the highest blood pressure 340 and the lowest blood pressure 350 may result in an insufficient number of samples.

Referring to FIG. 3B, a display that further displays an electrocardiogram (ECG) 360 according to an embodiment of the present disclosure is illustrated. Since the descriptions provided with reference to FIG. 3A apply to the oscillometric signal 310, the pressure signal 320, the Korotkoff sound 330, the highest blood pressure 340, and the lowest blood pressure 350 of FIG. 3B, detailed descriptions thereof are omitted.

The sphygmomanometer may simultaneously display two or more of the oscillometric signal 310, the Korotkoff sound 330, and the ECG 360. For example, referring to FIG. 3B, the sphygmomanometer may simultaneously display the oscillometric signal 310, the Korotkoff sound 330, and the ECG 360. The sphygmomanometer may display two or more of the oscillometric signal 310, the Korotkoff sound 330, and the ECG 360 in real time.

In this case, the oscillometric signal 310, the Korotkoff sound 330, and the ECG 360 may be synchronized with each other and may be displayed on the display. For example, the sphygmomanometer may display the oscillometric signal 310, the Korotkoff sound 330, and the ECG 360 that are measured at the same time on the display.

In other words, the sphygmomanometer may display, on the display, two or more of the oscillometric signal 310 sensed from the examinee while the internal pressure of the cuff that surrounds a body part of the examinee varies, the Korotkoff sound 330 included in a sound signal collected from a microphone while the internal pressure of the cuff varies, and the ECG 360 measured from the examinee while the internal pressure of the cuff varies. The two or more signals displayed on the display may be displayed based on the same time axis.

According to an embodiment, the sphygmomanometer may receive, from a separate ECG measuring device, the ECG 360 measured while the internal pressure of the cuff varies and may display the ECG 360 on the display. In addition, according to an embodiment, while the internal pressure of the cuff varies, the sphygmomanometer may measure and display the ECG 360 on the display.

Since the irregular pulse wave is most closely related to the ECG 360, the ECG 360 may be used to verify the irregular pulse wave that the sphygmomanometer determines using the oscillometric signal 310.

Hereinafter, the interval information of the oscillometric signal sensed from the examinee is described.

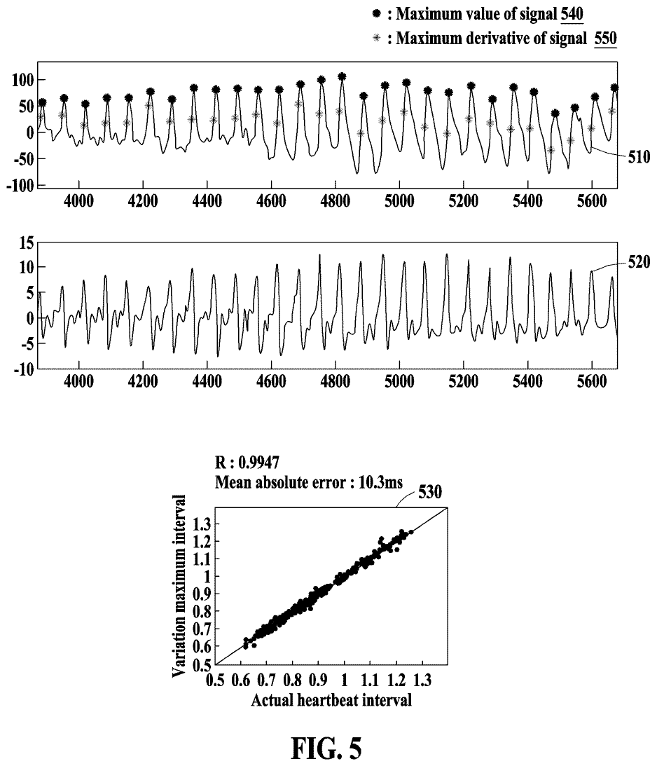

FIGS. 4 and 5 are diagrams of interval information of an oscillometric signal according to an embodiment of the present disclosure.

Referring to FIG. 4, a first graph 410 that displays only an oscillometric signal and a second graph 420 that displays a peak interval of an oscillometric signal are illustrated.

A sphygmomanometer may obtain interval information from the time when the magnitude of the oscillometric signal exceeds a threshold. Accordingly, an oscillometric signal 411 shown in the first graph 410 may be the oscillometric signal 411 displayed from the time when the magnitude thereof exceeds the threshold. The threshold may be referred to as a first threshold to distinguish it from a threshold for determining an irregular pulse wave described below.

Referring to the first graph 410, the oscillometric signal 411 is illustrated. The interval information of the oscillometric signal 411 may be required to determine whether an irregular pulse wave is present using the oscillometric signal 411.

The interval information of the oscillometric signal 411 may be intervals (in other words, peak intervals) which are a time period between an occurrence of a peak 413 of one signal of the oscillometric signal 411 and an occurrence of a peak 415 of a subsequent signal. The interval information of the oscillometric signal 411 may be intervals (in other words, valley intervals) from an occurrence of a valley 417 of one signal of the oscillometric signal 411 to a valley 419 of a subsequent signal.

Herein, the sphygmomanometer may determine whether an irregular pulse wave is present in the oscillometric signal 411 based on the peak interval or the valley interval. Accordingly, the magnitude of the peak or valley may not be considered in determining whether the irregular pulse wave is present.

Referring to the graph 420, a peak interval is illustrated. The second graph 420 may be a peak interval of the oscillometric signal 411 shown in the first graph 410. Accordingly, each point shown in the second graph 420 may indicate an interval between an occurrence of a peak of one signal and an occurrence of a peak of a subsequent signal. For example, a first point 421 may indicate that it took 0.48 seconds(s) from an occurrence of a peak of one signal to an occurrence of a peak of a subsequent signal.

The sphygmomanometer may determine whether an irregular pulse wave is present based on the interval information (in other words, the peak interval of the valley interval) of the oscillometric signal 411. The sphygmomanometer may calculate a mean value of intervals included in the interval information. When at least one interval that deviates from the mean value of the intervals by a threshold or more is present, the sphygmomanometer may determine that the irregular pulse wave is present. In this case, the threshold may be referred to as a second threshold to distinguish it from the first threshold with respect to the magnitude of the oscillometric signal described above.

For example, referring to the second graph 420, a mean value of cycles may be 0.4 s. Based on an assumption that the threshold is 25%, the sphygmomanometer may determine whether the irregular pulse wave is present based on whether a cycle that deviates from the range between 0.3 s and 0.5 s, which are ±25% of 0.4 s. Accordingly, since a second point 423, a third point 425, and a fourth point 427 deviate from the range between 0.3 s and 0.5 s, the sphygmomanometer may determine that the irregular pulse wave is present.

The threshold that serves as a criterion for determining whether the irregular pulse wave is present may be determined in various manners. For example, the threshold may be differently set depending on age and/or gender.

It is obvious to those skilled in the art that the description of the method of determining the irregular pulse wave using the peak interval described above may apply to the valley interval. Accordingly, the description of the method of determining the irregular pulse wave using the valley interval is omitted.

Referring to FIG. 5, an oscillometric signal 510, a graph 520 that differentiates an oscillometric signal, and a graph 530 are illustrated. FIG. 4 describes whether an irregular pulse wave is present using a cycle between maximum values 540 (in other words, a peak) of a signal of the oscillometric signal 510.

The most accurate method of determining whether an irregular pulse wave is present may be a method using an ECG, which is an electrical activity state of the heart during a heartbeat cycle. Specifically, an R-R interval of the ECG may be used to determine whether the irregular pulse wave is present.

The graph 530 may be a graph that compares the R-R interval of the ECG with a peak interval of the oscillometric signal 510. Referring to the graph 530, the R-R interval of the ECG may be similar to the peak interval of the oscillometric signal 510.

Contraction of the heart may be highly related to the ECG. The oscillometric signal 510 may be related to the heartbeat and may be sensed after the heart contracts. Accordingly, a signal of the oscillometric signal 510 may be sensed later than a corresponding signal of the ECG. A maximum derivative 550 of an oscillometric signal may be a moment at which the oscillometric signal most significantly increases and may occur before a maximum value 540 of the oscillometric signal. Referring to the oscillometric signal 510, in one signal, the maximum derivative 550 of the oscillometric signal may occur temporally earlier than the maximum value 540 of the oscillometric signal. In conclusion, a time when the maximum derivative 550 of the oscillometric signal occurs may be closer to a time when a peak (in other words, R of QRS waves) of the ECG than a time when the maximum value 540 of the oscillometric signal occurs.

Accordingly, the peak interval of the graph 520 that differentiates the oscillometric signal may be more similar to the R-R interval of the ECG than the peak interval of the oscillometric signal 510. Accordingly, the sphygmomanometer may more accurately determine whether the irregular pulse wave is present when using the peak interval or valley interval of the graph 520 that differentiates the oscillometric signal compared to when using the peak interval or valley interval of the oscillometric signal 510.

Therefore, the sphygmomanometer may determine whether the irregular pulse wave is present using the graph 520 that differentiates the oscillometric signal in addition to the oscillometric signal 510 as described with reference to FIG. 4. Since a method performed by the sphygmomanometer to determine whether the irregular pulse wave is present using a peak interval or a valley interval of the graph 520 that differentiates the oscillometric signal is the same as when using the oscillometric signal 510, the description is omitted.

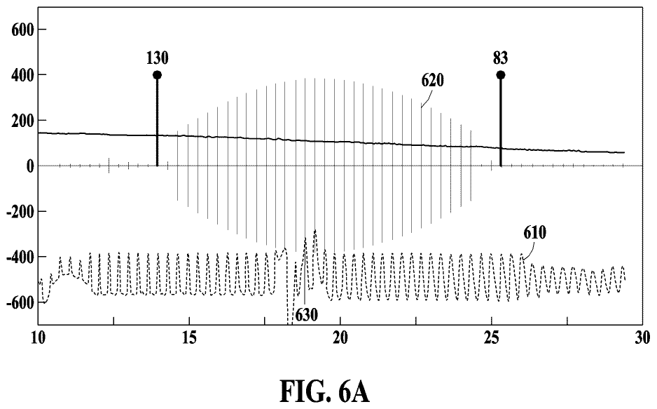

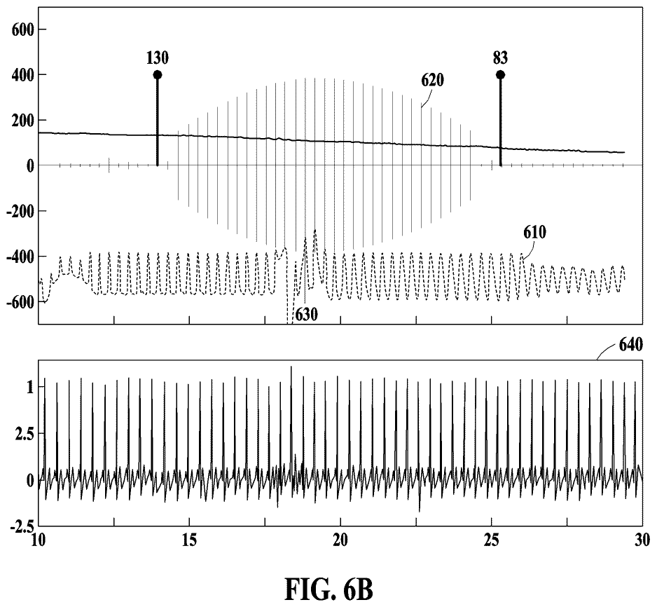

FIGS. 6A and 6B are diagrams of a measurement error according to an embodiment of the present disclosure.

Referring to FIG. 6A, an oscillometric signal 610 and a Korotkoff sound 620 are illustrated as shown in FIG. 3A.

When the oscillometric signal 610 is sensed from an examinee, the oscillometric signal 610 that is inaccurate may be collected due to noise. For example, when the examinee moves while sensing the oscillometric signal 610, the oscillometric signal 610 may include noise. When the sphygmomanometer determines whether the irregular pulse wave is present using the oscillometric signal 610 including noise, an error which determines that noise is present by an impact of the noise even though the irregular pulse wave is not actually present may occur. Accordingly, to prevent the error, whether the irregular pulse wave is present may need to be determined by excluding a portion corresponding to the noise from the oscillometric signal 610 including the noise or the oscillometric signal may need to be measured again.

Hereinafter, a method of determining whether noise occurs according to a movement of an examinee is described.

Referring to the oscillometric signal 610, since a shape of the graph in the middle portion is different from shapes immediately before and after the middle portion, it may be identified that noise occurs due to the movement of the examinee while measuring the blood pressure.

The sphygmomanometer may use the magnitude (e.g., a peak value and/or a valley value) of the signal to determine whether the noise occurs, unlike the method of determining whether the irregular pulse wave is present. In other words, the sphygmomanometer may determine a reference line for the peak or valley value of the oscillometric signal 610. When there is an interval in which a peak value or a valley value deviates from the reference line by a threshold or more, the sphygmomanometer may not determine whether a pulse wave is present in the interval.

Alternatively, when there is an interval in which a peak value or a valley value deviates from the reference line by the threshold, the sphygmomanometer may measure the blood pressure again. In this case, the threshold may be referred to as a third threshold to distinguish it from the first and second thresholds described above.

The reference line may be determined based on various criteria. According to an embodiment, the reference line for the peak value may be determined based on n (n is a natural number) peak values that occurred before a peak value 630 of a signal subject to determination. The reference line for the peak value may be a mean value of n (n is a natural number) peak values that occurred before the peak value 630 of the signal subject to determination. Accordingly, when the peak value 630 of the signal subject to determination deviates from the mean value of n peak values by the third threshold or more, the sphygmomanometer may determine that the interval including the peak value 630 of the signal subject to determination is an interval including noise.

According to an embodiment, the reference line for the peak value is a type of trend line. The sphygmomanometer may obtain interval information from a peak value of the oscillometric signal 610 that exceeds the first threshold. In this case, the sphygmomanometer may generate a trend line of peak values from the peak value of the oscillometric signal 610 that exceeds the first threshold. When the peak value 630 of the signal subject to determination deviates from the trend line by the third threshold or more, the sphygmomanometer may determine that the interval including the peak value 630 of the signal subject to determination is an interval including noise.

It is obvious to those skilled in the art that the method of determining the reference line for the peak value and determining whether the interval in which the peak value deviates from the reference line by the third threshold or more is present may apply to the valley value. Accordingly, a description of a method of determining a reference line for a valley value and determining whether an interval in which the valley value deviates from the reference line by the third threshold or more is present is omitted.

Referring to FIG. 6B, a display that further displays an ECG 640 according to an embodiment of the present disclosure is illustrated. Since the descriptions provided with reference to FIG. 3B may apply to a description related to the display of the ECG 640, a detailed description thereof is omitted. Since the descriptions provided with reference to FIG. 6A may apply to the oscillometric signal 610, the Korotkoff sound 620, and the peak value 630 of the signal, detailed descriptions thereof are omitted.

As described above, the interval including the peak value 630 of the signal may be an interval in which noise occurs due to the movement of the examinee while measuring the blood pressure. Referring to the ECG 640, a signal in an interval corresponding to the interval including the peak value 630 of the signal may fluctuate more than other signals. This may be caused by noise due to the movement of the examinee while measuring the blood pressure. However, the interval information of the ECG 640 may not change.

Hereinafter, operations of the sphygmomanometer when it is determined that an irregular pulse wave is present in an oscillometric signal is described.

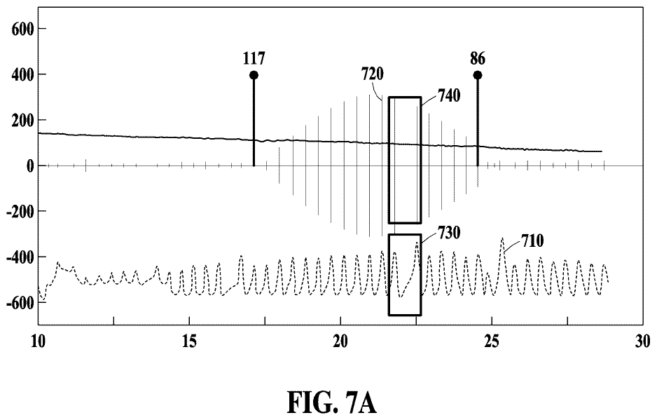

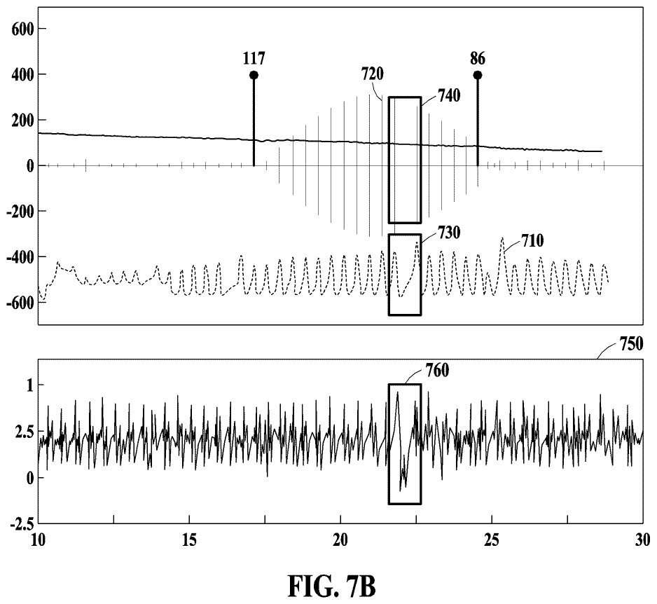

FIGS. 7A and 7B are diagrams of displaying an irregular pulse wave detected from a signal sensed from an examinee according to an embodiment of the present disclosure.

Referring to FIG. 7A, an oscillometric signal 710 and a Korotkoff sound 720 are illustrated. The oscillometric signal 710 may be a signal determined that an irregular pulse wave is present.

In other words, the oscillometric signal 710 may be determined that the irregular pulse wave is present based on the interval information. The oscillometric signal 710 may be determined that there is a cycle that deviates from a mean value of cycles corresponding to a peak interval or a valley interval by the second threshold or more. Alternatively, the oscillometric signal 710 may be determined that there is a cycle that deviates from a mean value of cycles corresponding to a peak interval or a valley interval of a graph that differentiates the oscillometric signal 710 by the second threshold or more.

The sphygmomanometer may display a visual mark 730 for an area determined to be the irregular pulse wave. The sphygmomanometer may allow the examinee to intuitively recognize that the irregular pulse wave has occurred by displaying the visual mark 730 on the area determined to be the irregular pulse wave. According to an embodiment, the sphygmomanometer may display the oscillometric signal 710 and the Korotkoff sound 720 included in the area determined to be the irregular pulse wave as bold lines, in different colors, or with blinking. Accordingly, a box mark on the area determined to be the irregular pulse wave in FIG. 7A is an example of the visual mark, and the present disclosure is not limited thereto.

In addition, according to an embodiment, the sphygmomanometer may display an area determined to be the irregular pulse wave in the oscillometric signal 710 and the Korotkoff sound 720 as a single visual mark. For example, the sphygmomanometer may display the area determined to be the irregular pulse wave in the oscillometric signal 710 and the Korotkoff sound 720 by using a single integrated visual mark.

In addition, the sphygmomanometer may simultaneously display a visual mark 730 on the area determined to be the irregular pulse wave in the oscillometric signal 710 and a visual mark 740 on a corresponding area of the Korotkoff sound 720. The visual mark 730 of the oscillometric signal 710 may be the same as or different from the visual mark 740 of the Korotkoff sound 720.

The oscillometric signal 710 and the Korotkoff sound 720 may be signals sensed simultaneously during the same time period. Accordingly, the oscillometric signal 710 and the Korotkoff sound 720 are arranged vertically for visual convenience in FIG. 7A and may share the same x-axis (the time axis). Accordingly, in the same time interval, the oscillometric signal 710 and the Korotkoff sound 720 may correspond to each other.

Therefore, it may be identified that the irregular pulse wave has occurred in the area of the Korotkoff sound 720 corresponding to the area determined to be the irregular pulse wave in the oscillometric signal 710.

In addition, when the sphygmomanometer determines that the irregular pulse wave is present in the oscillometric signal 710, the sphygmomanometer may determine whether the irregular pulse wave is caused by an arrhythmia. When the sphygmomanometer determines that the irregular pulse wave is present in the oscillometric signal 710, the sphygmomanometer may input the interval information to a machine learning model for determining atrial fibrillation and a machine learning model for determining premature contraction. The machine learning model for determining atrial fibrillation may be a machine learning model trained by various known methods and may determine whether the irregular pulse wave is caused by atrial fibrillation when the machine learning model receives the interval information of the oscillometric signal 710 in which the irregular pulse wave occurs. Similarly, the machine learning model for determining premature contraction may be a machine learning model trained by various known methods and may determine whether the irregular pulse wave is caused by premature contraction when the machine learning model receives the interval information of the oscillometric signal 710 in which the irregular pulse wave occurs. In this case, the premature contraction may include ventricular premature contraction, atrial premature contraction, and ventricular/atrial premature contraction.

When the irregular pulse wave is caused by premature contraction and/or atrial fibrillation of the arrhythmia, the sphygmomanometer may additionally display an alert in addition to the visual marks 730 and 740. The sphygmomanometer may suggest the examinee receive medical treatment because the irregular pulse wave has occurred and the arrhythmia is suspected of causing the irregular pulse wave. Through this, the sphygmomanometer may help the examinee to discover arrhythmias early.

Referring to FIG. 7B, a display that further displays an ECG 750 according to an embodiment of the present disclosure is illustrated. Since the oscillometric signal 710, the Korotkoff sound 720, and the visual marks 730 and 740 are described above, detailed descriptions thereof are omitted. In addition, since the description provided with reference to FIG. 3B may apply to the display of the ECG 750, a detailed description thereof is omitted.

The sphygmomanometer may further display the ECG 750. Referring to an area of the ECG 750 corresponding to the area determined to be the irregular pulse wave in the oscillometric signal 710, it may be identified that one cycle is omitted. In other words, referring to the ECG 750, the sphygmomanometer may verify whether the area determined to be the irregular pulse wave in the oscillometric signal 710 actually corresponds to an irregular pulse wave.

When there is no irregular pulse wave in the area of the ECG 750 corresponding to the area determined to be the irregular pulse wave in the oscillometric signal 710, the sphygmomanometer may determine that the area is not the irregular pulse wave. Conversely, when the irregular pulse wave is present in the area of the ECG 750 corresponding to the area that is not determined to be the irregular pulse wave in the oscillometric signal 710, the sphygmomanometer may determine the area to be the irregular pulse wave.

When the sphygmomanometer further displays the ECG 750, the sphygmomanometer may additionally display a visual mark 760 on the area of the ECG 750 corresponding to the area determined to be the irregular pulse wave in the oscillometric signal 710. Since the descriptions of the visual marks 750 and 760 apply to the visual mark 760, a detailed description thereof is omitted.

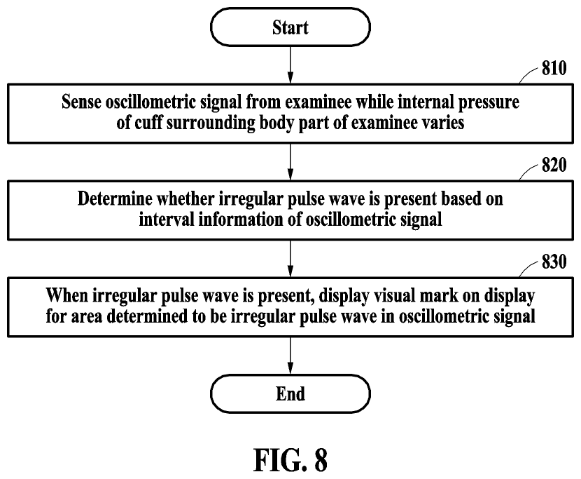

FIG. 8 is a flowchart of an operating method of a sphygmomanometer according to an embodiment of the present disclosure.

In the following embodiments, operations may be performed sequentially but not necessarily. For example, the order of the operations may change, and at least two of the operations may be performed in parallel. The operations shown in FIG. 2 may be performed by at least one component of the sphygmomanometer.

In operation 810, the sphygmomanometer may sense an oscillometric signal from an examinee while internal pressure of a cuff that surrounds a body part of the examinee varies.

In operation 820, the sphygmomanometer may determine whether an irregular pulse wave is present based on interval information of the oscillometric signal.

The sphygmomanometer may determine a reference line for a peak value or a valley value of the oscillometric signal and when there is an interval in which the peak or valley value deviates from the reference line by a threshold or more, the sphygmomanometer may not determine whether the irregular pulse wave is present in the interval.

In operation 830, when the irregular pulse wave is present, the sphygmomanometer may display a visual mark on a display for an area determined to be the irregular pulse wave in the oscillometric signal.

The sphygmomanometer may obtain interval information based on cycles corresponding to the peak interval or the valley interval of the oscillometric signal.

The sphygmomanometer may obtain the interval information based on cycles corresponding to a peak interval or a valley interval of a graph that differentiates the oscillometric signal.

When there is a cycle that deviates from a mean value of the cycles by the threshold or more, the sphygmomanometer may determine that the irregular pulse wave is present.

When the irregular pulse wave is present, the sphygmomanometer may determine whether the irregular pulse wave is caused by an arrhythmia. When the irregular pulse wave is caused by the arrhythmia, the sphygmomanometer may further display an alert on the display.

The sphygmomanometer may determine whether the irregular pulse wave is caused by atrial fibrillation of the arrhythmia by inputting the interval information to a machine learning model for determining atrial fibrillation.

The sphygmomanometer may determine whether the irregular pulse wave is caused by premature contraction of the arrhythmia by inputting the interval information to a machine learning model for determining premature contraction.

The sphygmomanometer may display a Korotkoff sound included together with the oscillometric signal on the display, wherein the Korotkoff sound is included in a sound signal collected by a microphone while the internal pressure of the cuff varies. The sphygmomanometer may display a visual mark on an area of the Korotkoff sound corresponding to the area determined to be the irregular pulse wave in the oscillometric signal.

Since the descriptions provided with reference to FIGS. 1 to 7B apply to each operation shown in FIG. 8, a detailed description thereof is omitted.

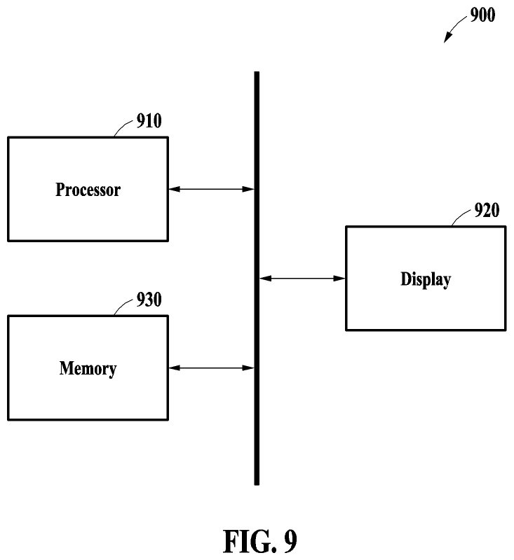

FIG. 9 is a diagram of a sphygmomanometer according to an embodiment of the present disclosure.

Referring to FIG. 9, a sphygmomanometer 900 may include a processor 910. In addition, the sphygmomanometer 900 may further include a display 920 and a memory 930. The processor 910, the display 920, and the memory 930 may communicate with each other via a bus, peripheral component interconnect express (PCIe), and/or a network on a chip (NoC).

The processor 910 may display, on the display, an oscillometric signal sensed from an examinee while internal pressure of a cuff that surrounds a body part of the examinee varies, may determine whether an irregular pulse wave is present based on interval information of the oscillometric signal, and when the irregular pulse wave is present, may display, a visual mark on the display for an area determined to be the irregular pulse wave in the oscillometric signal.

The processor 910 may display a Korotkoff sound together with the oscillometric signal on the display, wherein the Korotkoff sound is included in a sound signal collected by a microphone while the internal pressure of the cuff varies and may display a visual mark on an area of the Korotkoff sound corresponding to the area determined to be the irregular pulse wave in the oscillometric signal.

The processor 910 may obtain interval information based on cycles corresponding to a peak interval or a valley interval of the oscillometric signal.

The processor 910 may obtain the interval information based on cycles corresponding to a peak interval or a valley interval of a graph that differentiates the oscillometric signal.

When there is a cycle that deviates from a mean value of the cycles by a threshold or more, the processor 910 may determine that the irregular pulse wave is present.

The processor 910 may determine a reference line for a peak value or a valley value of the oscillometric signal and when there is an interval in which the peak or valley value deviates from the reference line by a threshold or more, the sphygmomanometer may not determine whether the irregular pulse wave is present in the interval.

When the irregular pulse wave is present, the processor 910 may determine whether the irregular pulse wave is caused by an arrhythmia, and when the irregular pulse wave is caused by the arrhythmia, the processor 910 may display an alert on the display.

The processor 910 may determine whether the irregular pulse wave is caused by atrial fibrillation of arrhythmia by inputting the interval information to a machine learning model for determining atrial fibrillation.

The processor 910 may determine whether the irregular pulse wave is caused by premature contraction of arrhythmia by inputting the interval information to a machine learning model for determining premature contraction.

The processor 910 may display, on the display, an oscillometric signal sensed from an examinee while internal pressure of a cuff that surrounds a body part of the examinee varies, may determine whether an irregular pulse wave is present based on interval information of the oscillometric signal, and when the irregular pulse wave is present, may display an alert on the display by determining whether the irregular pulse wave is caused by an arrhythmia.

The display 920 may display the oscillometric signal and the Korotkoff sound sensed from the examinee in real time while the internal pressure of the cuff that surrounds a body part of the examinee varies, based on the control of the processor 910. The display 920 may display visual marks on the oscillometric signal and the area of the Korotkoff sound where the irregular pulse wave is present.

The memory 930 may include computer-readable instructions. The processor 910 may perform the operations described above when the instructions stored in the memory 930 are executed by the processor 910. The memory 930 may be a volatile memory or a non-volatile memory.

In addition, the sphygmomanometer 900 may process the operations described above.

The method according to embodiments may be written in a computer-executable program and may be implemented as various recording media such as magnetic storage media, optical reading media, or digital storage media.

Various techniques described herein may be implemented in digital electronic circuitry, computer hardware, firmware, software, or combinations thereof. The implementations may be achieved as a computer program product, for example, a computer program tangibly embodied in a machine readable storage device (a computer-readable medium) to process the operations of a data processing device, for example, a programmable processor, a computer, or a plurality of computers or to control the operations. A computer program, such as the computer program(s) described above, may be written in any form of a programming language, including compiled or interpreted languages, and may be deployed in any form, including as a stand-alone program or as a module, a component, a subroutine, or other units suitable for use in a computing environment. A computer program may be deployed to be processed on one computer or multiple computers at one site or distributed across multiple sites and interconnected by a communication network.

Processors suitable for processing of a computer program include, by way of example, both general and special purpose microprocessors, and any one or more processors of any kind of digital computer. Generally, a processor will receive instructions and data from a read-only memory or a random-access memory, or both. Elements of a computer may include at least one processor for executing instructions and one or more memory devices for storing instructions and data. Generally, a computer also may include, or be operatively coupled to receive data from or transfer data to, or both, one or more mass storage devices for storing data, e.g., magnetic, magneto-optical disks, or optical disks. Examples of information carriers suitable for embodying computer program instructions and data include semiconductor memory devices, e.g., magnetic media such as hard disks, floppy disks, and magnetic tape, optical media such as compact disk read only memory (CD-ROM) or digital video disks (DVDs), magneto-optical media such as floptical disks, read-only memory (ROM), random-access memory (RAM), flash memory, erasable programmable ROM (EPROM), or electrically erasable programmable ROM (EEPROM). The processor and the memory may be supplemented by, or incorporated in special purpose logic circuitry.

In addition, non-transitory computer-readable media may be any available media that may be accessed by a computer and may include both computer storage media and transmission media.

Although the present specification includes details of a plurality of specific embodiments, the details should not be construed as limiting any invention or a scope that can be claimed, but rather should be construed as being descriptions of features that may be peculiar to specific embodiments of specific inventions. Specific features described in the present specification in the context of individual embodiments may be combined and implemented in a single embodiment. On the contrary, various features described in the context of a single embodiment may be implemented in a plurality of embodiments individually or in any appropriate sub-combination. Furthermore, although features may operate in a specific combination and may be initially depicted as being claimed, one or more features of a claimed combination may be excluded from the combination in some cases, and the claimed combination may be changed into a sub-combination or a modification of the sub-combination.

Likewise, although operations are depicted in a specific order in the drawings, it should not be understood that the operations must be performed in the depicted specific order or sequential order or all the shown operations must be performed in order to obtain a preferred result. In specific cases, multitasking and parallel processing may be advantageous. In a specific case, multitasking and parallel processing may be advantageous. In addition, it should not be understood that the separation of various device components of the aforementioned embodiments is required for all the embodiments, and it should be understood that the aforementioned program components and apparatuses may be integrated into a single software product or packaged into multiple software products.

The embodiments disclosed in the present specification and the drawings are intended merely to present specific examples in order to aid in understanding of the present disclosure, but are not intended to limit the scope of the present disclosure. It will be apparent to one of one of ordinary skill in the art that various modifications based on the technical spirit of the present disclosure, as well as the disclosed embodiments, can be made.

1. A sphygmomanometer comprising:
a processor configured to display, on a display, an oscillometric signal sensed from an examinee while internal pressure of a cuff that surrounds a body part of the examinee varies, determine whether an irregular pulse wave is present based on interval information of the oscillometric signal, and when the irregular pulse wave is present, display a visual mark on the display for an area determined to be the irregular pulse wave in the oscillometric signal.

2. The sphygmomanometer of claim 1, wherein the processor is configured to:
display a Korotkoff sound included in a sound signal collected by a microphone while the internal pressure of the cuff varies, together with the oscillometric signal on the display, and
display the visual mark on an area of the Korotkoff sound corresponding to the area determined to be the irregular pulse wave in the oscillometric signal.

3. The sphygmomanometer of claim 1, wherein the processor is configured to:
obtain the interval information based on cycles corresponding to a peak interval or a valley interval of the oscillometric signal.

4. The sphygmomanometer of claim 1, wherein the processor is configured to:
obtain the interval information based on cycles corresponding to a peak interval or a valley interval of a graph that differentiates the oscillometric signal.

5. The sphygmomanometer of claim 3, wherein the processor is configured to:
when a cycle that deviates from a mean value of the cycles by a threshold or more is present among the cycles, determine that the irregular pulse wave is present.

6. The sphygmomanometer of claim 1, wherein the processor is configured to:
determine a reference line for a peak value or a valley value of the oscillometric signal, and
when an interval in which the peak value or the valley value deviates from the reference line by a threshold or more is present, not determine whether the irregular pulse wave is present in the interval.

7. The sphygmomanometer of claim 1, wherein the processor is configured to:
when the irregular pulse wave is present, determine whether the irregular pulse wave is cause by an arrhythmia, and
when the irregular pulse wave is caused by the arrhythmia, display an alert on the display.

8. The sphygmomanometer of claim 7, wherein the processor is configured to:
determine whether the irregular pulse wave is caused by atrial fibrillation of the arrhythmias by inputting the interval information to a machine learning model for determining the atrial fibrillation.

9. The sphygmomanometer of claim 7, wherein the processor is configured to:
determine whether the irregular pulse wave is caused by premature contraction of the arrhythmias by inputting the interval information to a machine learning model for determining the premature contraction.

10. A sphygmomanometer comprising:
a processor configured to display, on a display, an oscillometric signal sensed from an examinee while internal pressure of a cuff that surrounds a body part of the examinee varies, determine whether an irregular pulse wave is present based on interval information of the oscillometric signal, and when the irregular pulse wave is present, display an alert on the display by determining whether the irregular pulse wave is caused by an arrhythmia.

11. A method of operating a sphygmomanometer, the method comprising:
displaying, on a display, an oscillometric signal sensed from an examinee while internal pressure of a cuff that surrounds a body part of the examinee varies;
determining whether an irregular pulse wave is present based on interval information of the oscillometric signal; and
when the irregular pulse wave is present, displaying a visual mark on the display for an area determined to be the irregular pulse wave in the oscillometric signal.

12. The method of claim 11, further comprising:
displaying a Korotkoff sound together with the oscillometric signal on the display, wherein the Korotkoff sound is included in a sound signal collected by a microphone while the internal pressure of the cuff varies, wherein the display of the visual mark on the display comprises:
displaying the visual mark on an area of the Korotkoff sound corresponding to the area determined to be the irregular pulse wave in the oscillometric signal.

13. The method of claim 11, further comprising:
obtaining the interval information based on cycles corresponding to a peak interval or a valley interval of the oscillometric signal.

14. The method of claim 11, further comprising:
obtaining the interval information based on cycles corresponding to a peak interval or a valley interval of a graph that differentiates the oscillometric signal.

15. The method of claim 13, wherein the determining of whether the irregular pulse wave is present comprises:
when a cycle that deviates from a mean value of the cycles by a threshold or more is present among the cycles, determining that the irregular pulse wave is present.

16. The method of claim 11, wherein the determining of whether the irregular pulse wave is present comprises:
determining a reference line for a peak value or a valley value of the oscillometric signal, and
when an interval in which the peak value or the valley value deviates from the reference line by a threshold or more is present, not determining whether the irregular pulse wave is present in the interval.

17. The method of claim 11, further comprising:
when the irregular pulse wave is present, determining whether the irregular pulse wave is cause by an arrhythmia; and
when the irregular pulse wave is caused by the arrhythmia, displaying an alert on the display.

18. The method of claim 17, wherein the determining of whether the irregular pulse wave is caused by the arrhythmia comprises:
determining whether the irregular pulse wave is caused by atrial fibrillation of the arrhythmias by inputting the interval information to a machine learning model for determining the atrial fibrillation.

19. The method of claim 17, wherein the determining of whether the irregular pulse wave is caused by the arrhythmia comprises:
determining whether the irregular pulse wave is caused by premature contraction of the arrhythmias by inputting the interval information to a machine learning model for determining the premature contraction.

20. A sphygmomanometer comprising:
a processor configured to synchronize and display, on a display, two or more of an oscillometric signal sensed from an examinee while internal pressure of a cuff that surrounds a body part of the examinee varies, a Korotkoff sound included in a sound signal collected by a microphone while the internal pressure of the cuff varies, and an electrocardiogram measured from the examinee while the internal pressure of the cuff varies.
